(19) United States
(12) Patent Application Publication
(10) Pub. No.: US 2019/0344073 A1
(43) Pub. Date: Nov. 14, 2019

(54) METHOD AND SYSTEM FOR TINNITUS SOUND THERAPY

(71) Applicant: Otoharmonics Corporation, Portland, OR (US)

(72) Inventors: Michael John Baker, Portland, OR (US); Brenda Edin, Portland, OR (US); Sudeshna Dutta, Portland, OR (US); Leonardo Martinez Hornak, Ciudad de la Costa (UY); Leonardo Raul Cecilia Delgado, Montevideo (UY)

(21) Appl. No.: 16/405,886

(22) Filed: May 7, 2019

Related U.S. Application Data
(60) Provisional application No. 62/669,283, filed on May 9, 2018.

(51) Int. Cl. A61N 1/36; H04R 25/00; A61N 1/372

(52) U.S. Cl. CPC A61N 1/361; A61N 1/37247; H04R 25/75

(57) ABSTRACT

Methods and systems are provided for a system for making, administering, reviewing, and displaying tinnitus sound therapies. A graphical user interface provided on a user device provides a snapshot of the changes in the user's tinnitus therapy sound enabling better tracking of the evolution of the sound. A patient may be able to track their progress, improving the likelihood of them continuing with the therapy, and the overall success of the therapy.

### CROSS REFERENCE TO RELATED APPLICATIONS

The present application claims priority to U.S. Provisional Application No. 62/669,283, entitled “METHOD AND SYSTEM FOR TINNITUS SOUND THERAPY”, and filed on May 9, 2018. The entire contents of the above-listed application are hereby incorporated by reference for all purposes.

### FIELD

The present description relates generally to methods and systems for tinnitus sound therapy including a graphical user interface for generating and displaying data pertaining to a tinnitus sound therapy.

### BACKGROUND AND SUMMARY

Tinnitus is the sensation of hearing sounds when there are no external sounds present and can be loud enough to attenuate the perception of outside sounds. Tinnitus may be caused by inner ear cell damage resulting from injury, age-related hearing loss, and exposure to loud noises. The tinnitus sound perceived by the affected patient may be heard in one or both ears and may include ringing, buzzing, clicking, and/or hissing sounds.

Some methods of tinnitus therapy include producing a sound in order to mask the tinnitus of the patient. One example is shown by U.S. Pat. No. 7,850,596 wherein the masking treatment involves an algorithm that modifies a sound similar to a patient's tinnitus sound in a predetermined manner. Data from a hearing test is stored as an audiogram and used by a healthcare provider during the tinnitus therapy.

However, the inventors herein have recognized that it may be difficult for a patient to understand how their therapy is progressing. Even for a healthcare professional, analyzing the audiogram may require substantial time and training, making consultations lengthy and tedious. Lacking awareness on the progress of their therapy, a patient may feel discouraged and may not adhere to the therapy, resulting in ineffective treatment.

The inventors herein have thus recognized that it may be advantageous to provide a user interface to the patient and/or the healthcare professional for graphically displaying information about the current progress of the therapy. The displayed information may provide a snapshot of the therapy and its progress, enabling both the patient and the healthcare professional to review if the therapy is nearing completion. In addition, the healthcare professional may be able to quickly infer salient features of the therapy, allowing them to easily gather data on the case. The interface may also help patients to stay on course and adhere to the therapy for effective treatment results. One example approach includes displaying a graphical user interface including a visual representation of real-time changes to a therapy sound presented to a user during a tinnitus therapy, the real-time changes based on user input. For example, the data may be presented in the graphical user interface as a graph or pie chart indicating the composition (e.g., percentage) each sound utilized in the tinnitus therapy, as well as the intensity of each sound in the therapy. As one example, each sector of the pie chart may have a distinct color indicative of the different sound (e.g., white noise, chirping noise, hissing noise, etc.) applied, with a tonal property of the color (e.g., degree of darkness) adjusted to be proportional to the corresponding sound intensity. At each therapy session, a new pie chart may be created, with updates in each sector, enabling progress of the therapy to be monitored. The technical effect of displaying therapy data in the form of an easily comprehensible graph is that tinnitus treatment sessions may be tracked and reviewed by both the patient and the healthcare professional on a user device. This may allow for more effective treatment, such as due to improved patient cooperation.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows an example system for tinnitus therapy including a patient device and a healthcare professional device in communication with a central server.

FIG. 2 shows an example graphical user interface that may be displayed on a patient device and/or healthcare professional device of the tinnitus therapy system.

FIG. 3 shows additional example GUIs that may be displayed on the patient device and/or healthcare professional device of the tinnitus therapy system

FIG. 4 method for generating a tinnitus therapy sound and displaying it on a graphical user interface.

FIGS. 5-6 show an example sound survey that may be used during a tinnitus therapy.

FIG. 7 shows an example method for updating a graphical user interface during a tinnitus therapy.

FIG. 8 shows an adjustment to a graphical user interface during a tinnitus therapy responsive to user input.

### DETAILED DESCRIPTION

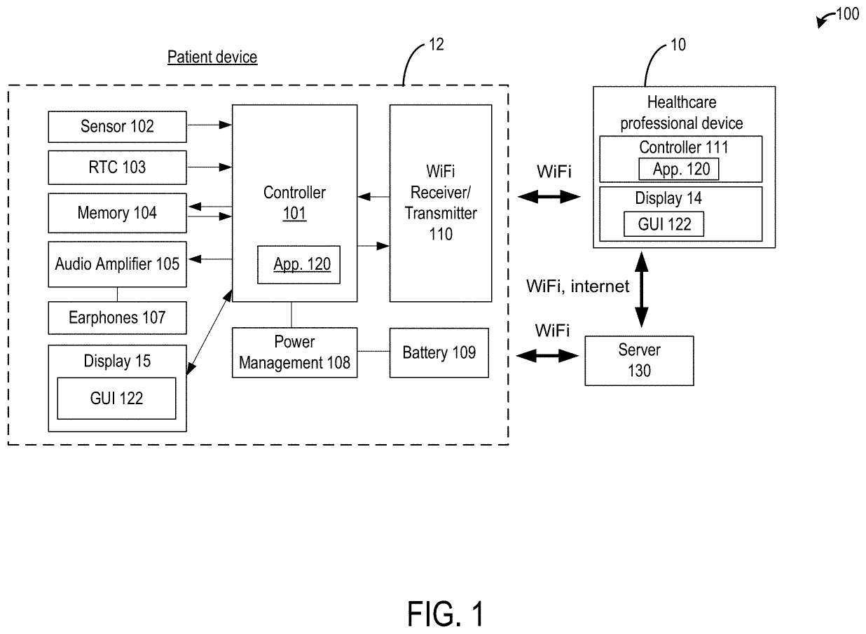

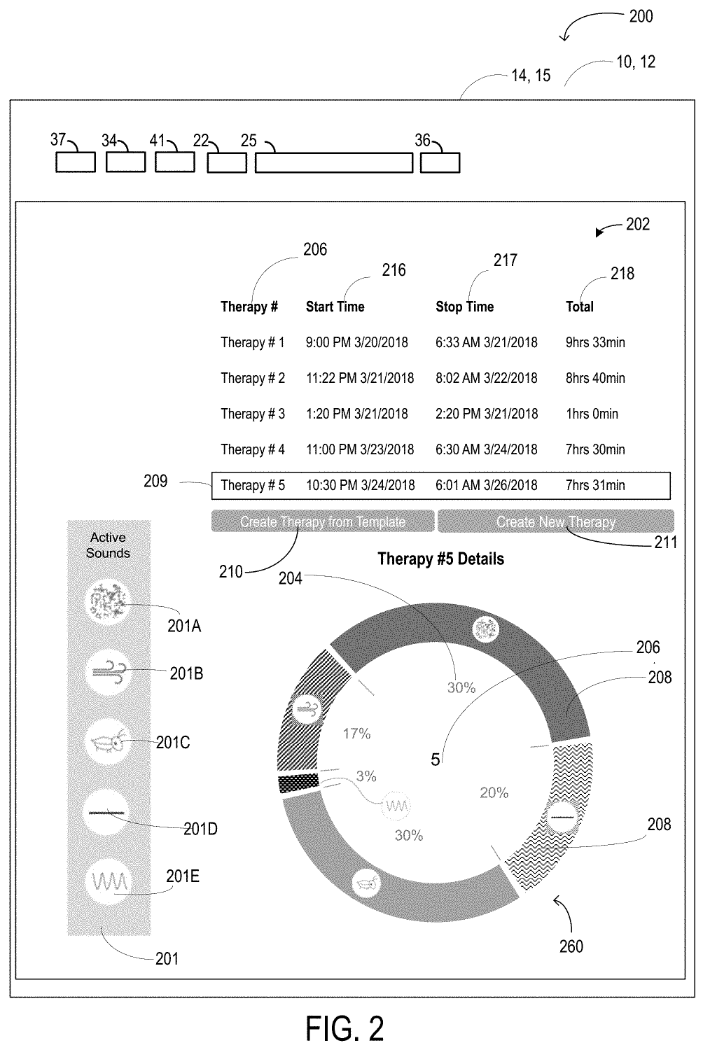

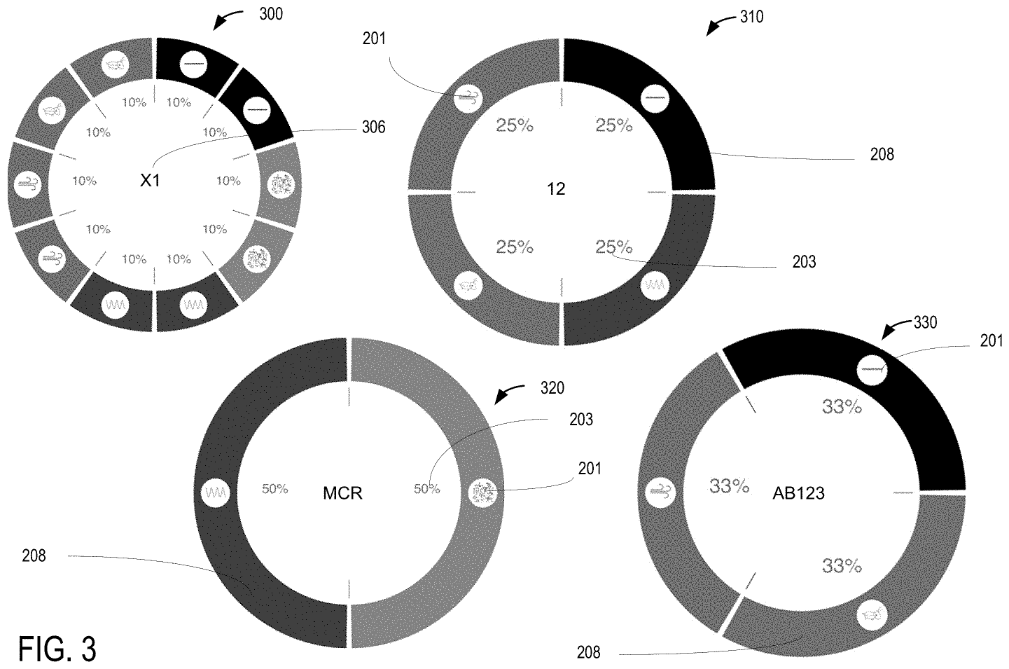

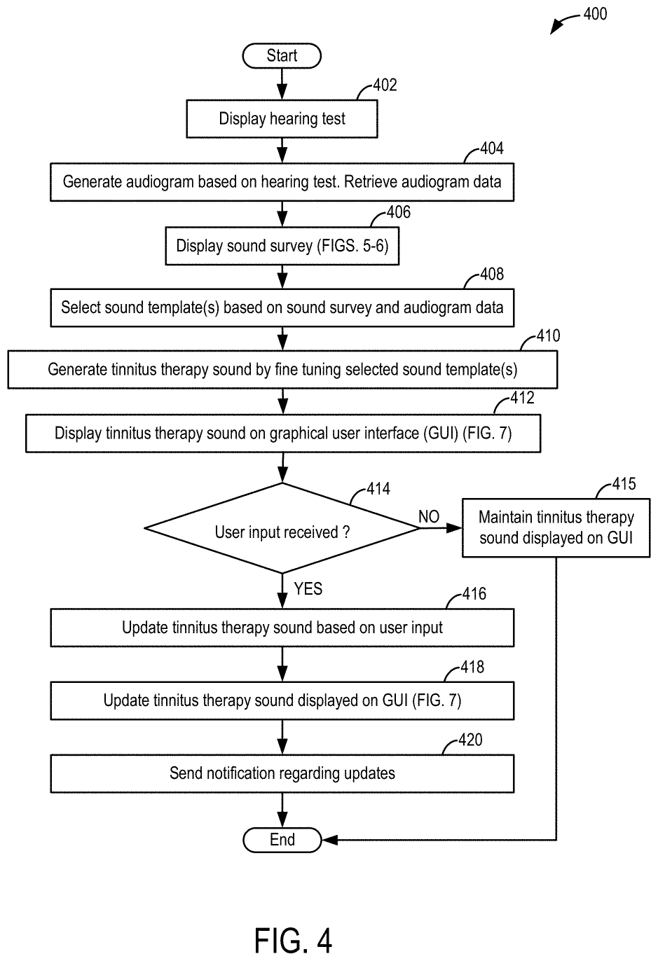

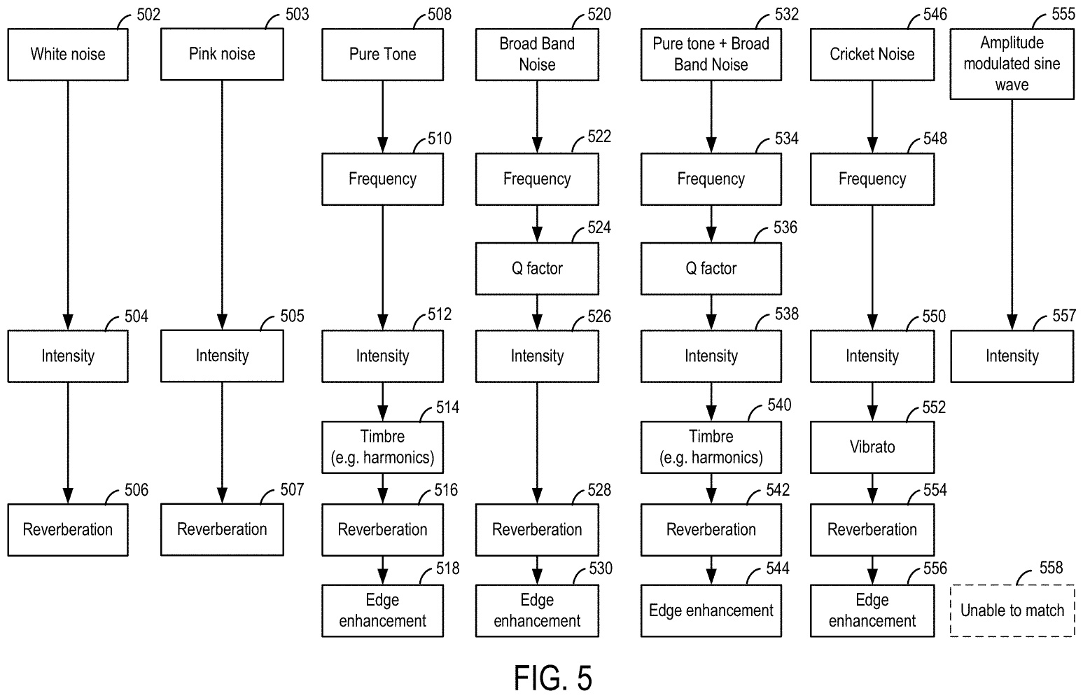

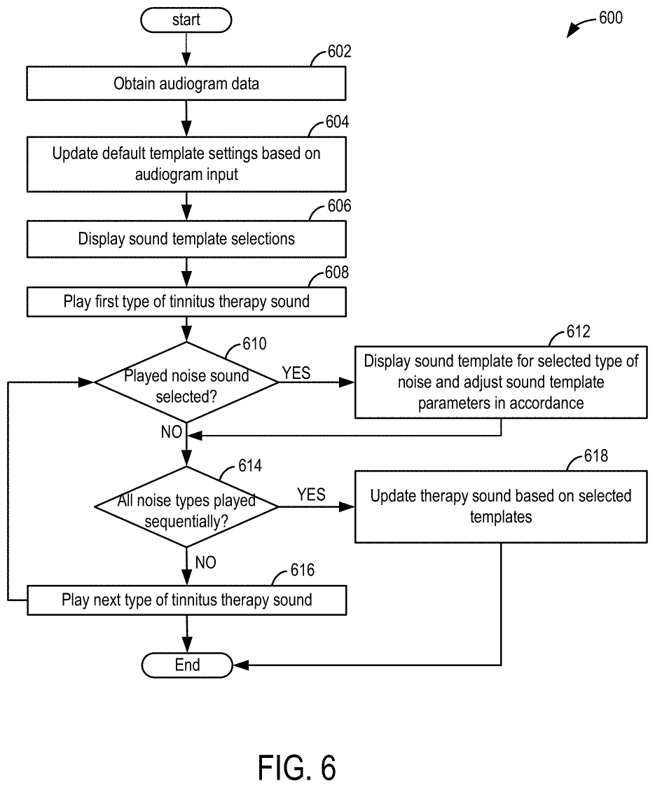

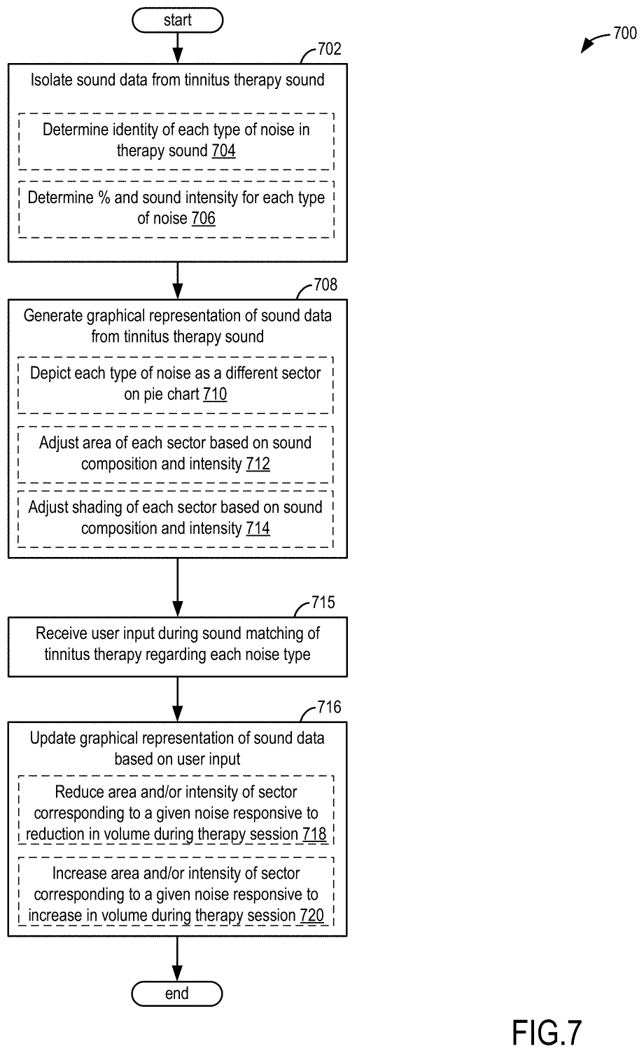

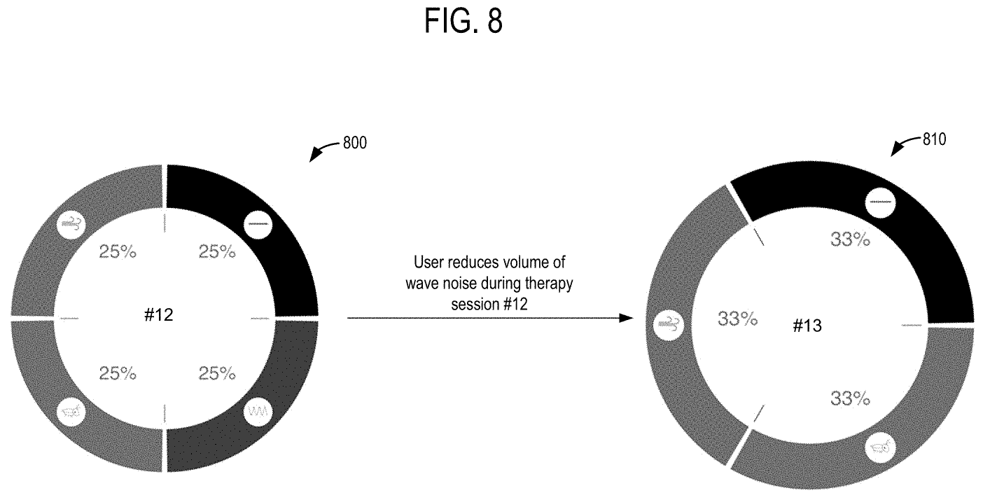

Methods, systems, and interfaces are provided for tinnitus therapy generation, tracking, displaying, and reviewing. The methods, systems, and interfaces described herein may be adapted for other audio therapies or neurological disorders and treatments. FIG. 1 shows an example system for tinnitus therapy wherein therapy sessions are generated by a controller and carried out on a patient's device, the therapy sessions tracked and the progress displayed via a graphic user interface in communication with both the patient device and a healthcare professional device. FIG. 4 shows an example method for tinnitus therapy including displaying progress of a tinnitus therapy on a graphical user interface, such as an interface of FIGS. 2-3. FIG. 5 shows an example sound survey and FIG. 6 shows a method for establishing sound template parameters based on the sound survey. FIG. 7 shows a method for populating and updating a GUI based on user input. FIG. 8 shows an example updating of a GUI responsive to user input received on a therapy session.

Tinnitus therapy for a patient may include a tinnitus therapy sound generated via a healthcare professional's device. The tinnitus therapy sound may be based on and include one or more types of sounds. For example, different types of sounds such as white noise, pink noise, pure tone, broad band noise, and cricket noise may be included in the tinnitus therapy sound. Specific tinnitus therapy sounds, or sound templates, may be pre-determined and may include a white noise sound, a pink noise sound, a pure tone sound, a broad band noise sound, a cricket noise sound, an amplitude modulated sine wave, and/or a combined tone sound. A user may be presented with one or more of the above tinnitus therapy sound templates via the healthcare professional's device. Using a plurality of user interfaces of the healthcare professional's device, a user may select and modify one or more tinnitus therapy sound templates in order to generate a tinnitus therapy sound similar to the user's perceived tinnitus. However, the modifications may not include adding further amplitude of frequency modulation to the templates. In one example, a user may include a medical provider such as a physician, nurse, technician, audiologist, or other medical personnel. In another example, the user may include a patient.

Referring to FIG. 1, a schematic diagram of a system 100 for tinnitus therapy including hardware and hardware connectivity is shown. System 100 includes example devices for a tinnitus therapy including a healthcare professional's device 10 and a patient's device 12. Healthcare professional's device 10 may be used and/or operated by a medical provider including, but not limited to, physicians, audiologists, nurses, and/or technicians. In another example, healthcare professional's device 10 may be used and/or operated by a patient. Thus, the user of the healthcare professional's device may be one or more of a patient or a medical provider. Further, the user of the patient's device 12 may be the patient.

Healthcare professional's device 10 and patient's device 12 are physical, non-transitory devices configured to hold data and/or instructions executable by a logic subsystem. The logic subsystem may include individual components that are distributed throughout two or more devices, which may be remotely located and/or configured for coordinated processing. One or more aspects of the logic subsystem may be virtualized and executed by remotely accessible networked computing devices. Healthcare professional's device 10 and patient's device 12 may be configured to execute one or more instructions related to a tinnitus therapy. A detailed example embodiment of a patient device and a healthcare professional device is shown with reference to FIG. 2.

Healthcare professional's device 10 and patient's device 12 may generate tinnitus therapy sound templates and tinnitus therapy sounds to transmit the generated electronic tinnitus therapy to the user. In one example, healthcare professional's device 10 and patient's device 12 may interact via a wired or wireless network which may allow for bidirectional communication between the devices. In another example, a patient's device 12 may track and/or record tinnitus therapy data, including metadata that may be transmitted to the healthcare professional's device 10. In another example, recorded and/or stored therapy data may be written in an HTML5 format such that the transferred data, via a remote portal, may be received on a secured webpage. Furthermore, the recorded and/or stored therapy data, in the form of raw data as well as in the form of a graphical user interface, may be transferred from each device to a central server 130, and likewise may be retrieved onto either device from the central server.

Patient device 12 may include a controller 101 that executes instructions stored on memory 104 for enabling a method of tinnitus treatment on the patient device. Patient device 12 is powered using electrical power drawn from battery 109, the power managed via a power management system 108. Power management system 108 may be configured to adjust the power consumption of device 12 based on operator usage. For example, power consumption may be reduced when the device is not in use or when the device is in a “sleep” mode. The power management system may include a charge regulator, a coulomb counter, and/or main power regulators. Device 12 may include an audio amplifier 105 for adjusting an audio output generated during a tinnitus therapy. This may include adjusting an audio intensity, volume, frequency, or other audio parameter. The audio output may be generated via the execution of instructions stored on memory 104, which may be non-transitory memory, relating to a tinnitus therapy method may be transmitted from the patient device 12 to a patient via an external device, such as earbuds 107. The earbuds may be coupled to device 12 via a communication channel which may be include wired communication, wirelessly communication (e.g., Wi-Fi or Bluetooth communication). The wireless communication may be optionally selected by a user via a button arranged on the patient device 12, wherein the device 12 may provide feedback regarding an activation or a deactivation of the wireless communication in the form of sounds and/or lights. The audio amplifier 105 may receive instructions from the controller 101 to adjust a volume output of the earphones 107, for example. The instructions may be based on prior therapy sessions, stored biometric data, and/or user inputs received via a user interface, herein shown as display 15. A transmitter/receiver 110 of the device may enable communication via the wireless communication channels. One or more sensors 102 may be coupled to the device for sensing various user parameters. The device may further include a real-time clock (RTC 103) or monitoring a duration of execution of a tinnitus therapy, which may include monitoring durations of specific sounds, user responses, and the like.

Patient's device 12 may include a set of customized earphones 107. In one example, the earphones 107 comprise a medical grade silicon and are bespoke to a patient's ears. Further, earphones 107 may be used while generating a tinnitus therapy via a healthcare professional's device as well as during the tinnitus therapy via the patient's device. In another example, another type of earphones or listening device may be used during generating the tinnitus therapy and during listening to the generated tinnitus therapy (e.g., tinnitus sound match). In some examples, a different set of earphones may be used while generating the tinnitus therapy via the healthcare professional's device 10 than when listening to the generated tinnitus therapy via the patient's device 12.

A storage device of controller 101 may store application data to enable an application 120 that connects to a cloud-based health care server 130 and/or collects information for transmission to the cloud-based server. The application may also retrieve information gathered by device sensors 102, input devices (e.g., display 15 and other forms of user interface such as a mouse, keypad, etc.), and other devices in communication with the patient device (e.g., earbuds 107 connected via a Bluetooth link), etc.

Display 15 may include one or more buttons allowing a user to modify controller operating parameters. For example, the buttons on display 15 may allow the user (e.g., the patient) to adjust operation of the device 12. For example, the user may play and pause audio, adjust volume settings, and connect to Wi-Fi. The user is also able to turn off the device 12 via buttons on the display 106. In some examples, a standby feature may be incorporated. The controller 101 may include instructions for executing the standby feature wherein the real-time clock tracks a duration of time that audio output pertaining to the tinnitus therapy has been paused. If the duration of time is greater than a threshold pause duration (e.g., 15 minutes), then the controller may turn off the device without instructions from the user including reducing power usage via the power management system. In this way, the patient device 12 may include physical buttons arranged along the device that enable a user to adjust items displayed on the display 15 and/or respond to prompts illustrated on the display 15. Additionally or alternatively, the buttons may be electronic buttons, wherein the display 15 comprises a touchscreen, and where the user may select one or more electronic buttons on the touchscreen to modify the display 15.

In some examples, the controller 101 may include instructions for playing a voice through the earphones alerting a user of a change in operating parameters. For example, the voice may be programmed to say, when appropriate, ‘Wi-Fi on’, ‘connected’, ‘Power off’, ‘battery low’, etc.

The real-time clock 103 enables the controller to track a duration of an ongoing activity. For example, the real-time clock may allow the controller to track a duration of a sleep cycle, duration of a therapy session, and/or provide time stamps regarding changes in device activity. Memory 104 enables the device to save therapy data, including biometric data and portions of a therapy session, for a threshold amount of time. For example, the threshold amount of time is 90 days. The data and other stored information may be erased from the memory following the earlier of the 90-day threshold being reached or the data being transmitted to an auxiliary device. This may ensure memory is available for future therapy sessions. The auxiliary device may be a separate hard drive. Additionally or alternatively, the auxiliary device may be a server or other device for saving the data to a memory cloud, wherein the data may be accessed and downloaded for analysis when desired.

Data from the device 10 may be transmitted to auxiliary devices via Wi-Fi. The auxiliary devices may include a computer, cell phone, tablet, or other computing device capable of connecting to Wi-Fi and storing data. The auxiliary devices may belong to the healthcare provider or the patient. In some examples, data is sent to auxiliary devices belonging to both the healthcare provider and the patient. In this way, both the health care provider and the patient may access the patient's therapy session data sets.

The connection between the patient device 12 and the auxiliary device or healthcare professional device 10 may be mediated through a web application software. The software may be a “class A-no injury or damage to health is possible” form of software. The software is downloaded and/or installed onto personal computers, tablets, and/or mobile devices readily available to the health care provider and patient. Additionally or alternatively, the software may be accessed from personal computers without download. As such, the software may be accessed via the internet as a web interface. In some examples, additionally or alternatively, the web interface may be accessed from a personal computer, smartphone, cellular device, tablet, or the like. Additionally or alternatively, the software may be downloadable as an application to a cellular device, smartphone, tablet, or the like, wherein the application and/or web interface may be in communication with each of the application on the patient device and the application on the healthcare professional device. In one example, the application and/or software may be streamable to a plurality of wireless, internet-enabled devices including hearing aids and the like. The software includes a user interface, an HTML/Javascript, angular+libraries, and server API module. The user interface may further include modules on the software configured to allow the patient to review their treatment progress, communicate with their health care provider, and select different tinnitus sound matches. The application may provide an interface to allow the patient to monitor their treatment. Usage data includes treatment duration, when the treatment was played, any adjustments to the amplitude, and the battery state and the beginning and end of the therapy. The Patient App uses a login which is authenticated by the server. The patient logs in with a unique user ID and password. Once authenticated, the patient only has access to their own session data. All server functions will be accessed via the Server API Module.

In some examples, the application 120 on the patient device is distinct from the application on the healthcare professional device, although they may be the same. The Application 120 on the patient device may be a web based single page application using HTML and JavaScript in the browser and using the Server 130 API to communicate with the back end. The Server API Module provides an encapsulation of the server functions in a convenient form. It provides a JavaScript API and communicates to the server via TLS using RESTful interface calls. Parameters are validated where possible. The HTML/JavaScript layer uses a number of components, such as AngularJS, and supporting components to provide a single page web application framework.

The healthcare provider (HCP) device 12 may use a secure TLS connection to a server to provide, generate, and refine therapies for a patient, and to provide information on therapy usage by the patient. All server functions may be accessed via a server API module. HCPs login with a unique user ID and password. The HCP can only access and modify information for their own patients. Sound match generation and control may be executed through HTTP with encrypted payload requests to the earphones 107, which may be wireless earbuds in one example.

The Provider App is a web based single page application using HTML and JavaScript in the browser and using the Server API to communicate with the back end. The Server API Module provides an encapsulation of the server functions in a convenient form. It provides a JavaScript API and communicates to the server via TLS using RESTful interface calls. Parameters are validated where possible. The HTML/JavaScript layer uses a number of components, such as AngularJS, and supporting components to provide a single page web application framework. The Provider App may play a Sound Match for 5 minutes. This may mitigate confusion between the Provider App and the Patient App.

One or more sensors 102 may be located in one or more of the earphones. The sensors are configured to monitor biometric data of the patient. In one example, the patient device 12 may include a band to enable the device to be worn around a neck of the patient and rest atop the patient's shoulders. The earphones are then inserted into each of the patient's ears. As such, sensors located in the earbuds may gather different biometric data than sensors located in the band of the patient device.

Like display screen 15 of patient device 12, healthcare professional device 10 may also include a display screen 14 for displaying information to the user and receiving digital information from the user, such as patient information and adjustments to the tinnitus therapy. In one example, the display screen(s) may be a touch screen. Information received from the user (which may be the patient or the healthcare professional) may be in various digital forms that represent a user's inputs. For example, the user may enter text, select, and/or move slide bars or other adjustable input buttons. In the example of the display screen being a touch screen, the user may adjust the input buttons through the touch screen. In another example, if the display screen is not a touch screen, the user may adjust the input buttons through a secondary device such as a computer mouse and/or keyboard.

Display screen 14 of healthcare professional's device 10 may include a plurality of input buttons for selecting sound parameters, such as frequency, intensity, octaves, Q factor, reverberation, and/or white noise edge enhancement.

Application 122 running on the patient device 12 may enable a graphical user interface (GUI) 122 to be generated for display on the patient device 12, such as on display 15 (which may be, for example, a touch screen). The graphical user interface 122 displays various details regarding a patient's tinnitus therapy to the patient on the patient device 12. These may include, for example, a current tinnitus sound therapy being provided to the patient, changes since a last therapy, progress made since a last therapy, etc. In addition, during the course of a tinnitus therapy, any adjustments made by the patient to the sounds of the therapy may be updated at the GUI. For example, if a user increases the volume or intensity of a particular type of sound (e.g., pink noise) during a therapy session, the GUI 122 may be adjusted to reflect the change. The update is communicated to a central server 130 which also automatically updates a GUI 122 displayed to a healthcare provider on a healthcare professional device 10.

Healthcare professional device 10 may similarly include a controller 111. A storage device of the controller 111 may store application data as instructions in non-transitory memory of the controller 111 that when executed enable application 120. The application 120 may connect to the cloud-based health care server 130 and/or collect information for transmission to the cloud-based server. The application may also retrieve information gathered from patient device 12, including data gathered via by device sensor(s) 102, devices in communication with the patient device (e.g., earbuds 107 connected via a Bluetooth link), as well as input received via a user interface of the healthcare professional device 10, such as on display 14, which may be, for example, a touch screen. The application 120 may also enable a graphical user interface (GUI) 122 to be generated for display on the healthcare professional device 10 (herein also referred to as therapist device). The graphical user interface 122 displays various details regarding a patient's tinnitus therapy to the healthcare provider on device 10. These may be the same details also displayed to the patient. These may include, for example, a current tinnitus sound therapy being provided to the patient, changes since a last therapy, a composition of the current sound therapy, progress made since a last therapy, etc. In essence, the GUI 122 provides a synopsis and/or brief summary of the therapy to both the patient and the healthcare provider. This allows the patient to track their progress, and makes the patient more likely to follow through with the therapy. The GUI also enables a healthcare provider to quickly come up to speed about the current status of the patient's therapy as well as the therapy history (e.g., progress made, sounds previously used, etc.). Based on the details displayed via the GUI 122, a healthcare professional may make updates and changes to a tinnitus sound therapy for the patient. All changes to a GUI 122, including those made by a patient via patient device 12, or a healthcare provider via device 10, may be updated to a central server 130 and communicated to both devices 10, 12. Example GUIs are shown with reference to FIGS. 2-3

While not shown, it will be appreciated that the various components shown with reference to the patient device 12 may also be included in the healthcare professional device 10. That is, each of device 10, 12 may include sensors, batteries, clocks, audio amplifiers, power management systems, etc.

FIG. 2 shows an example graphical user interface 200 that may be displayed on one or both of a healthcare professional device and a patient device of a tinnitus therapy system. In one example, the devices include devices 10, 12 of FIG. 1. Each of the devices may be running an application to generate the graphical user interface (GUI) 200. The GUI 200 is displayed on a user interface, such as a touch-screen display of the device. For example, the display may include display screens 14, 15 of devices 10, 12, respectively, of FIG. 1. The GUI displays details pertaining to a tinnitus therapy being prescribed by the healthcare professional to the patient.

The tinnitus therapy sound generated with the methods described herein may also be referred to herein as a tinnitus sound match or a tinnitus therapy sound match. Various control buttons may be included on the patient device display screen 15 and/or the healthcare device display screen 14 for generating the tinnitus therapy. The controls used for generating the tinnitus sound match may include, as non-limiting examples, a tinnitus sound match input button 37, generating a tinnitus therapy via therapy input button 34, copying a tinnitus sound match via copy tinnitus sound match input button 41, and adding a template to the tinnitus therapy via add template input button 22. The tinnitus therapy sound may be generated based on adjustments to pre-defined tinnitus therapy templates, the pre-defined tinnitus therapy templates including a tinnitus therapy sound or combination of sounds (e.g., cricket noise, broad band noise, pure tone and broad band noise, etc.) within certain frequency and intensity ranges. The pre-defined tinnitus therapy templates may be modified by patient-specific hearing threshold data such that the tinnitus therapy sound template includes a tinnitus therapy sound audible to the patient.

GUI 200 is presented on devices 10, 12 as a dashboard depicting details pertaining to a particular therapy session. The GUI 200 may be retrieved on the devices 10, 12 at least at the start of a therapy session. The GUI 200 includes a table 202 depicting therapy history and details of a current therapy session 209. Details may include a therapy number 206 associated with the therapy session, each consecutive therapy session assigned a consecutive number (e.g. starting from a first session indicated as Therapy #1). The therapy number enables the patient and the healthcare professional (hereafter also referred to as therapist) to track progress of the therapy. For example, a patient may be able to infer how far they are in their therapy and estimate approximately how many more sessions are likely to be required (or how many sessions they have already completed). This helps to encourage the patient and decreases the likelihood that they will discontinue in the midst of a therapy. A duration 218 of therapy session 209 including a starting date and time 216 and a stopping date and time 217 may be tracked, such as via a real-time clock of the device. In the depicted example, all therapy sessions conducted thus far are listed with the most recent (or current) therapy session highlighted. However, in other examples, only the current therapy session may be displayed and the user may retrieve the particulars of all previous sessions via a recall history button.

GUI 200 may further include a chart 260, herein displayed as a ring or donut chart. In other examples, chart 260 may be a pie chart or other graphical display (e.g., a bar graph). Chart 260 includes details of the highlighted or selected therapy session 209, displayed as sectors 208. In particular, the specific sound composition of each therapy session is depicted as sectors, each sector 208 representing a different sound in the therapy sessions. The areas of each sector 208 may be proportional the relative percentage (%) 204 of intensity of each individual sound making up the tinnitus therapy. Active sounds used in any given therapy session may include, as non-limiting examples, white noise, pure tone, modulated pure tone, band noise, and insect noise. Each noise is depicted by a representative symbol, color, or design. A legend 201 listing the available active sounds 201A-E and their representative symbol, color, or design may be displayed on the GUI 200, alongside the chart 260, for quick and easy reference. Symbols corresponding to the different active noises 201A-E may be pre-determined by the developer of the graphical user interface or may be selected from a list of available symbols by the patient or healthcare professional. The sound symbols may also be displayed on top of the corresponding sectors 208.

In still further examples, an intensity of each active sound in the total sound of the therapy session may be indicated by the intensity of the color of the corresponding sector. Therein, as the intensity of a given active sound increases, the color of a corresponding sector increases. A color gradient legend may be included on the interface together with the chart 260 to provide a reference of each darkness level with intensity.

In the depicted example, active noise 201A (e.g., white noise) accounts for 30% of the sound of the current therapy session 209 (therapy session #5). Other active noise contributions include 20% noise contribution from hissing noise 201B, 30% noise contribution from cricket noise 201C, 3% noise contribution from pure tone noise 201D, and 17% noise contribution from wave noise 201E.

The graphical user interface 200 may further display a button 210 to create a tinnitus therapy from a template where the patient or the healthcare professional may choose a template or a combination of templates. Example templates and generation of templates is elaborated at FIGS. 5-6. The graphical user interface 200 may further display a button 211 that allows the patient or healthcare professional to create a new therapy, where the new therapy is not generated from templates. In one example, the template may differ from the non-template in that the template may comprise a fixed combination of active sounds, wherein only the contribution (e.g., percentage) of each active sound may be adjusted. The non-template may be blank, such that a combination of active sounds and the contribution of each is selected by one or more of the patient and healthcare professional. In some examples, the newly created therapy from the non-template may be saved and stored as a template.

At the onset of the therapy session 209, the therapist may generate a new therapy (e.g., from a known template) causing a more detailed chart of the current therapy to be displayed on the graphical user interface 200 of both the patient device 12 and the therapist device 10.

In one example, once a tinnitus therapy sound template is selected, specific tinnitus therapy sound template displays may also be displayed on the graphical user interface in order to enable a user to generate a specific tinnitus therapy, or tinnitus therapy sound. Each tinnitus therapy sound template display may include a specific tinnitus therapy sound template (e.g., cricket noise, broad band noise, etc.), along with various input buttons to adjust sound parameters of the tinnitus therapy sound template. Example tinnitus therapy sound templates, and their associated displays, may include a cricket noise sound template, a white noise sound template, a pure tone sound template, and/or a broad band noise sound template. In addition, a tinnitus therapy sound template display may include a set of controls and/or adjustments for modifying the sound characteristics of the tinnitus therapy sound template. The controls and/or adjustments may include a volume adjustment (e.g. intensity adjustment), a frequency adjustment (e.g., pitch adjustment), a timbre adjustment, a Q factor adjustment, a vibrato adjustment, a reverberation adjustment, and/or a white noise edge enhancement adjustment. As such, the controls and/or adjustments of a template may include an input button and/or slide bar input.

Graphical user interface 200 may also include a session notes window 25 that includes a space to input notes about a tinnitus therapy. Notes written in the session notes window 25 may be displayed as part of the tinnitus therapy. Additional buttons or windows may adjust various therapy parameters, such as to provide a help-to-sleep option, a changing volume option, and a maximum duration option. Further, a sound option may enable the physician to allow adjustment of the volume of the generated tinnitus sound match on the patient's device 12. For example, a patient may be able to adjust his/her tinnitus therapy volume during the duration of the tinnitus therapy treatment.

In order to complete the tinnitus therapy, when selected, an end session input button 36, or similar input button, saves the tinnitus therapy to both the healthcare professional's device 10 and the patient's device 12. In one example, once the therapy is completed and the session ends, a patient's device 12 is connected to healthcare professional's device 10 and the tinnitus therapy is loaded onto patient's device 12. In another example, after completing the tinnitus therapy on the healthcare professional's device 10, the completed tinnitus therapy (or tinnitus therapy sound) may be e-mailed over a secure network which may then be accessed via an internet connection on the patient's device 12. In yet another example, the competed tinnitus therapy sound may be transferred between the healthcare professional's device 10 and the patient's device 12 by bidirectional communication via a wired connection or a portable storage device, or via a server where all the data is stored.

Turning now to FIG. 3, several additional example embodiments of a graphical user interface, displayed as a chart, are shown. It will be appreciated that the graphical user interface may include other forms of data representation, such as a pie chart, a bar graph, etc. Each of the charts 300-330 represents a distinct tinnitus sound therapy comprising different compositions of sounds. Each therapy may be indicated by a therapy number 306. Therapy numbers 306 may be numbers, letters or combinations thereof indicating the specific tinnitus sounds generated during the execution of a tinnitus therapy method, such as method 400 of FIG. 4. As with FIG. 2, the color and symbol 201 associated with each sector 208 is representative of a distinct active noise and the area of each sector is representative of a contribution of that noise in the total sound applied during the therapy.

Each of the charts 300, 310, 320, and 330 may represent different templates used to perform a tinnitus therapy. In the example of FIG. 3, each of the sectors 208 for each individual chart 300, 310, 320, and 330 is equally sized such that each active noise is incorporated equally. However, it will be appreciated that each sector 208 may be sized differently, as shown in FIG. 2, such that each active noise is not contributed to the therapy evenly.

The charts 300, 310, 320, and 330 differ from one another in a number of active noises used to perform the tinnitus therapy. In the example of chart 300, ten active noises are used. In the example of chart 310, four active noises are used. In the example of chart 320, two active noises are used. In the example of chart 330, three active noises are used. In one example, the charts 300-330 may represent a progression of a patient's tinnitus therapy, wherein a first therapy session, which corresponds to the chart 300, is undesired or adjusted, resulting in a second therapy session represented by chart 310. Thus, chart 330 may represent a fourth therapy session wherein the patient and healthcare professional have added and eliminated active noises and adjusted a contribution of each of the retained active noises to provide a more desirable tinnitus therapy session.

Turning now to FIG. 4, an example method 400 is depicted for conducting a tinnitus therapy and displaying data pertaining to a therapy session to a user via a graphical user interface (GUI). Instructions for carrying out method 400 as well the other methods included herein may be executed by a controller based on instructions stored in a memory of the controller and in conjunction with signals received from the devices and sensors of the tinnitus therapy system, such as devices 10, 12 of FIG. 1. The controller may adjust sounds played on a device during a tinnitus therapy session and vary a n interface displayed on the device according to the methods described below.

At 402, the method includes displaying a hearing test. At 404, an audiogram is generated based on the hearing test. The controller may retrieve audiogram data from the audiogram. The audiogram input may include hearing threshold data determined during a patient audiogram. An individual patient's hearing threshold data may include decibel and frequency data. As such, the frequency, expressed in hertz (Hz), is the “pitch” of a sound where a high pitch sound corresponds to a high frequency sound wave and a low pitch sound corresponds to a low frequency sound wave. In addition, a decibel (dB) is a logarithmic unit that indicates the ratio of a physical quantity relative to an implied reference level such that the physical quantity is a sound pressure level. Therefore, the hearing threshold data is a measure of an individual patient's hearing level or intensity (dB) and frequency (Hz). The audiogram input and/or patient hearing data may be received by various methods. Based on a generated audiogram from the hearing test, a user may input hearing level and frequency data when prompted by the user interface. In yet another example, the audiogram input of patient hearing data may be uploaded to the healthcare professional's device via a wireless network, a portable storage device, or another wired device. In another example, the audiogram or patient hearing data may be input by the user (e.g., medical provider) with the user interface of the healthcare professional's device.

At 406, the method includes displaying a sound survey, as elaborated at FIGS. 5-6. Then, at 408, once the hearing threshold data from the audiogram has been received, and the sound survey data has been received, the method includes selecting one or more sound templates based on the sound survey. The method combines hearing threshold data with sound template data in order for the tinnitus therapy sound template to be in the correct hearing range of an individual patient. To be in a desired hearing range of an individual patient, specific frequency and intensity ranges may not be included in the tinnitus therapy sound template. Specifically, if an audiogram's hearing threshold data reflects mild hearing loss of a patient (e.g. 30 dB, 3,000 Hz), the frequency and intensity range associated with normal hearing will be eliminated from the template default settings (e.g. 0-29 dB; 250-2,000 Hz) such that a default setting starts at the hearing level of the patient. In one example, an audiogram may include a range of frequencies including frequencies at 125 Hz, 250 Hz, 500 Hz, 1,000 Hz, 2,000 Hz, 3,000 Hz, 4,000 Hz, 6,000 Hz, 8,000 Hz, 10,000 Hz, 12,000 Hz, 14,000 Hz, 15,000 Hz, and/or 16,000 Hz. In one example, the sound template is selected based on input received from the patient or health care provider (HCP). The controller may display sound templates to the user based on the hearing test and sound survey, and receive a user selection indication.

At 410, the method generates a tinnitus therapy sound. Once tinnitus therapy sounds are generated, the patient or healthcare provider may decide to further fine tune the tinnitus therapy sound if the sound generated does not resemble the perceived tinnitus. Fine tuning the tinnitus therapy sound may comprise further selecting additional sounds or deselecting already selected sounds. The sounds may be white noise, pink noise, pure tone, broad band noise, combined pure tone and broad band, cricket noise, or amplitude modulated sine wave. For each of the sounds, intensity, reverberation, frequency, timbre, Q factor, vibrato, and edge enhancement may be adjusted to help fine tune the tinnitus therapy sound at 410. Once the patient or healthcare professional determines that the tinnitus therapy sound resembles perceived tinnitus, the method confirms a tinnitus therapy sound and retrieves the associated data which may be used to generate a tinnitus sound therapy graphical user interface.

At 412, the method includes displaying the tinnitus therapy sound on a GUI. As elaborated with reference to FIG. 7, the displaying may include isolating sound composition and intensity data from a tinnitus therapy sound in order to determine the percentage (%) sound intensity for each sound in the therapy. The percent (%) of sound intensity for a particular sound displayed in the graphical user interface may be calculated based on the ratio of that particular sound intensity to the sum of all the sound intensities utilized in the tinnitus therapy. Each sound intensity and its calculated % sound intensity data may be converted into a sector of donut-shaped charts such that a summation of each sector completes the donut shape and is displayed on a user device.

At 414, it may be determined if the user input has been received, such as via a user device during a therapy session. For example, it may be determined if a user (patient or HCP) has adjusted (increased or decreased) the volume of a specific type of sound/noise during a therapy session. If not, at 415, the tinnitus therapy sound provided may be maintained and the corresponding display on the GUI may also be maintained. However, if user input is received, then at 416, the tinnitus therapy sound is updated based the user input. For example, if a user reduces the volume of a cricket noise, then the cricket noise composition of the tinnitus therapy sound is reduced. Additionally or alternatively, the change in the active sound may be evenly distributed among the other active sounds. For example, if the cricket noise is one of five active sounds, and the cricket noise is reduced 10%, then the four remaining active sounds may be increased by 2.5% each unless otherwise indicated via the user input. Thus, if the cricket noise is increased 10%, then the other active sounds may be decreased 2.5% each unless otherwise indicated via the user input.

At 418, the tinnitus therapy sound data displayed on the GUI is updated. For example, an area of the sector of the donut chart corresponding to the cricket noise may be reduced. As another example, a darkness, brightness, or translucency of the sector may be updated. An example updating method is shown at FIG. 7, and an example change in GUI responsive to user input is shown at FIG. 8. As such, during a tinnitus therapy, following sound matching, as a user listens to a therapy sound, the overall volume or amplitude or frequency of a given noise that matches their tinnitus sound is expected to reduce. This can be tracked by an HCP by receiving user input and tracking a change in the composition of the GUI of the therapy session over time.

At 420, a notification may be sent regarding the updating of the GUI. For example, a notification may be sent to both the patient and the HCP device saying that the most recently generated therapy sound has been updated.

Now referring to FIG. 5, an example method 500 for generating the sound survey, including adjusting tinnitus sound templates is shown. The sound survey may include inputting hearing threshold data determined by an audiogram and selecting tinnitus therapy sound templates in order to create a tinnitus therapy sound. As such, a tinnitus therapy sound template may be selected based on the similarity of the tinnitus therapy sound template (e.g. tinnitus sound type) to the patient's perceived tinnitus. The sound survey is an initial step in generating a tinnitus therapy sound such that the template(s) selected will be adjusted following the conclusion of the sound survey.

FIG. 5 shows example tinnitus therapy sound template selections including sound template adjustment parameters. Creating a tinnitus therapy may include presenting each of a white noise, a pink noise, a pure tone, a broad band noise, a combined pure tone and broad band noise, a cricket noise, and an amplitude modulated sine wave tinnitus therapy sound template to a user. In an alternate embodiment, creating a tinnitus therapy may include presenting a different combination of these sound templates to a user. For example, creating a tinnitus therapy may include presenting each of a white noise, a pink noise, a pure tone, a broad band noise, and a cricket noise tinnitus therapy sound template to a user. In yet another example, creating the tinnitus therapy may include presenting each of a white noise, a pure tone, and a combined tone tinnitus therapy sound template to a user. The combined tone may be a combination of at least two of the above listed sound templates. For example, the combined tone may include a combined pure tone and broad band noise tinnitus therapy sound template.

After playing each of the available tinnitus therapy sound templates, the user may select which sound type, or sound template, most resembled their perceived tinnitus. In this way, generating a tinnitus therapy sound may be based on the tinnitus therapy sound template selected by the user. After selecting one or more of the tinnitus therapy sound templates, the selected sound template(s) may be adjusted to more closely resemble the patient's perceived tinnitus. Adjusting the tinnitus therapy sound, or tinnitus therapy sound template, may be based on at least one of a frequency parameter and an intensity parameter selected by the user. As discussed above, a tinnitus therapy sound template(s) may be selected if the tinnitus therapy sound(s) resembles the perceived tinnitus sound of a patient. However, in one example, a patient's perceived tinnitus sound may not resemble any of the tinnitus therapy sound templates. As such, at 558, an unable to match input may be selected. Upon selection of an individual tinnitus therapy sound template, a tinnitus therapy sound template may include adjustment inputs including adjustments for frequency, intensity, timbre, Q factor, vibrato, reverberation, and/or white noise edge enhancement. The pre-determined order of adjustments of the tinnitus therapy sound template(s) selections are described below with regard to FIG. 5.

FIG. 5 begins at 502, by selecting a white noise sound template. White noise sound template adjustments may include, at 504, adjustments for intensity and adjustments for reverberation, at 506. For example, adjusting the tinnitus therapy sound may be first based on the intensity parameter and second based on a reverb input when the tinnitus therapy sound template selected by the user is the white noise tinnitus therapy sound template. If a pink noise template is selected at 503, the pink noise sound template may be adjusted based on intensity at 505 and reverberation at 507. Adjustments to the pink noise sound template may be similar to adjustments to the white noise sound template. For example, adjusting the tinnitus therapy sound may be first based on the intensity parameter and second based on a reverb input when the tinnitus therapy sound template selected by the user is the pink noise tinnitus therapy sound template. In another example, a pure tone sound template, at 508, may be selected. A pure tone sound template may be adjusted based on frequency, at 510, and intensity, at 512. In addition, a pure tone sound template may be further adjusted base on timbre, at 514. In one example, timbre may include an adjustment of the harmonics of a tinnitus therapy sound including an octave and/or fifth harmonic adjustments. Further, a pure tone sound template may be adjusted based on a reverberation, at 516, and a white noise edge enhancement, at 518. In one example, adjusting the tinnitus therapy sound may be first based on the frequency parameter, second based on the intensity parameter, third based on one or more timbre inputs, further based on a reverberation (e.g., reverb) input, and fifth based on an edge enhancement input when the tinnitus therapy sound template selected by the user is the pure tone sound template. In another example, a white noise edge enhancement may be a pre-defined tinnitus therapy sound template. Herein, a white noise edge enhancement sound template may be referred to as a frequency windowed white noise sound template. Additionally, a white noise edge enhancement adjustment may include adjusting the frequency windowed white noise based on an intensity input.

Continuing with FIG. 5, a broad band noise sound template, at 520, may be selected. A broad band noise sound template may include an adjustment for frequency, Q factor, and intensity, at 522, 524, and 526, respectively. Further adjustments to a broad band noise sound template may include reverberation, at 528, and white noise edge enhancement, at 530. For example, adjusting the tinnitus therapy sound may be first based on the frequency parameter, second based on a Q factor input, third based on the intensity parameter, fourth based on a reverberation input, and fifth based on an edge enhancement input when the tinnitus therapy sound template selected by the user is the broad band noise tinnitus therapy sound template.

At 532, a combination tinnitus sound template may be selected. A combination tinnitus sound template may include both a pure tone and a broad band noise sound. As such, the combination pure tone and broad band noise sound template may include adjustments for frequency, Q factor, and intensity, at 534, 536, and 538, respectively. A combination pure tone and broad band noise sound template may include further adjustments for timbre, reverberation, and white noise edge enhancement, at 540, 542, and 544, respectively. For example, adjusting the tinnitus therapy sound may be first based on the frequency parameter, second based on a Q factor input, third based on the intensity parameter, fourth based on a timbre input, fifth based on a reverberation input, and sixth based on an edge enhancement input when the tinnitus therapy sound template selected by the user is the combined pure tone and broad band noise tinnitus therapy sound template.

At 546, a cricket noise sound template may be selected. A cricket noise sound template may include adjustments for frequency, at 548, and intensity, at 550. Further adjustments to a cricket noise template may include a vibrato adjustment, at 552. A vibrato adjustment may include adjustment to the relative intensity of the cricket noise sound template. A cricket noise sound template may also include adjustments for reverberation, at 554, and white noise edge enhancement, at 556. For example, adjusting the tinnitus therapy sound may be first based on the frequency parameter, second based on the intensity parameter, third based on a vibrato input, fourth based on a reverberation input, and fifth based on an edge enhancement input then the tinnitus therapy sound template selected by the user is the cricket noise tinnitus therapy sound template.

At 555, an amplitude modulated sine wave sound template may be selected. In one example, the amplitude modulated sine wave template may include a base wave and carrier wave component. Additionally, the amplitude modulated sine wave template may include adjustments for intensity (e.g., amplitude) at 557, or alternatively adjustment to the base wave frequency. In alternate embodiments, additional or alternative adjustments may be made to the amplitude modulated sine wave sound template.

In another embodiment, the tinnitus therapy sound template(s) may include a plurality of tinnitus therapy sounds including but not limited to the tinnitus therapy sounds mentioned above with regard to FIG. 5. For example, FIG. 5 may include alternative or additional sound templates which may be displayed and played for the user. Specifically, in one example, an additional combination tinnitus sound template may be presented to and possibly selected by the user. In one example, the additional combination tinnitus therapy sound template may include a combined white noise and broad band noise sound template. In another example, the additional combination tinnitus therapy sound template may include a template combining more than two tinnitus therapy sound types.

It should be appreciated that once a user selects a sound template and its properties (such as intensity or frequency), no additional modulation is applied to the selection. Further it should be appreciated that once a user selects a sound level, treatment or therapy where the selected sound is replayed occurs at the selected sound level without lowering.

Referring now to FIG. 6, a method 600 begins at 602 by obtaining audiogram data via an audiogram input and/or patient hearing data. The audiogram input may include hearing threshold data. An individual patient's hearing threshold data may include decibel and frequency data. As such, the frequency, expressed in hertz (Hz), is the “pitch” of a sound where a high pitch sound corresponds to a high frequency sound wave and a low pitch sound corresponds to a low frequency sound wave. In addition, a decibel (dB) is a logarithmic unit that indicates the ratio of a physical quantity relative to an implied reference level such that the physical quantity is a sound pressure level. Therefore, the hearing threshold data is a measure of an individual patient's hearing level or intensity (dB) and frequency (Hz). Additionally, the audiogram input and/or patient hearing data may be received by various methods. Based on a generated audiogram from the hearing test, a user may input hearing level and frequency data when prompted by the user interface. In yet another example, the audiogram input of patient hearing data may be uploaded to the healthcare professional's device via a wireless network, a portable storage device, or another wired device. In another example, the audiogram or patient hearing data may be input by the user (e.g., medical provider) with the user interface of the healthcare professional's device.

At 604, default template settings are updated based on the audiogram input. Once the audiogram data has been received, the initial tinnitus therapy sound template settings (e.g. frequency and intensity) may be modified by the hearing threshold data from an individual patient's audiogram. For example, in order for the tinnitus therapy sound template to be in the correct hearing range of an individual patient, specific frequency and intensity ranges may not be included in the tinnitus therapy sound template. Specifically, if an audiogram's hearing threshold data reflects mild hearing loss of a patient (e.g. 30 dB, 3,000 Hz), the frequency and intensity range associated with normal hearing will be eliminated from the template default settings (e.g. 0-29 dB; 250-2000 Hz) such that a default setting starts at the hearing level of the patient. In one example, an audiogram may include a range of frequencies including frequencies at 125 Hz, 250 Hz, 500 Hz, 1,000 Hz, 2,000 Hz, 3,000 Hz, 4,000 Hz, 6,000 Hz, 8,000 Hz, 10,000 Hz, 12,000 Hz, 14,000 Hz, 15,000 Hz, and/or 16,000 Hz.

Additionally, the hearing threshold data from an individual patient's audiogram may be used to determine sensitivity thresholds (e.g. intensity and frequency) of the tinnitus therapy sound. For example, hearing threshold data may include maximum intensity and frequency thresholds for an individual patient such that the tinnitus therapy sound template's intensity and/or frequency may not be greater than a patient's sensitivity threshold. As such, the sensitivity levels will further limit the intensity and frequency range of the tinnitus therapy sound template. As such, the frequency and intensity range of the tinnitus therapy sound template may be based on the hearing level and hearing sensitivity of the patient. Therefore, at 604, the tinnitus therapy sound template(s) default settings are adjusted to reflect the audiogram, hearing threshold data, and hearing sensitivity of the patient.

At 606, a plurality of tinnitus therapy sound templates may be displayed. In one example, the tinnitus therapy sound templates may include tinnitus sounds including cricket noise, white noise, pink noise, pure tone, broad band noise, amplitude modulated sine wave sound, and a combination of pure tone and broad band noise. Specifically, each tinnitus therapy sound template may be pre-determined to include one of the above listed tinnitus sounds having pre-set or default sound characteristics or template settings (e.g., frequency, intensity, etc.). As described above, in other examples more or less than 6 different tinnitus therapy sound templates may be displayed.

At 608, the tinnitus therapy sound template selection process begins by playing pre-defined tinnitus therapy sounds (e.g., sound templates). In one example, the pre-defined tinnitus therapy sounds may be played in a pre-determined order including playing a white noise sound first followed by a pink noise sound, pure tone sound, a broad band sound, a combination pure tone and broad band sound, a cricket noise sound, and amplitude modulated sine wave sound. In another example, the tinnitus therapy sounds may be played in a different order. Further, the different tinnitus therapy sounds may either be presented/played sequentially (e.g., one after another), or at different times. For example, the sound templates may be grouped into sound categories (e.g., tonal or noise based) and the user may be prompted to first select between two sound templates (e.g., cricket and white noise). Based on the user's selection, another different pair of sound templates (or tinnitus therapy sounds) may be displayed and the user may be prompted to select between the two different sound templates. This process may continue until one or more of the tinnitus therapy sound templates are selected. In this way, the sound survey may narrow in on a patient's tinnitus sound match by determining the combination of sound templates included in the patient's perceived tinnitus sound.

For example, at 608, a first type of noise sound may be played. For example, the user may be presented, via a user interface (e.g., display and/or earphones) of the patient or healthcare professional's device, with a noise-based sound template and a tone-based sound template. The noise-based sound template may be a white noise sound template, a broad band noise sound template, a pink noise sound template, or some combination template of the white noise, broad band noise, and/or pink noise sound templates. The tone-based sound template may be a pure tone sound template, a cricket sound template, or some combined pure tone and cricket sound template.

At 610, the method includes determining if the noise-based sound was predominantly selected. In one example, the noise-based sound may be predominantly selected if an input selection of the noise-based sound is received. In another example, the user interface of the healthcare professional's device may include a sliding bar between the noise-based and tone-based sounds. In this example, the noise-based sound may be predominantly selected if an input (e.g., a sliding bar input) is received indicating the tinnitus sound is more like the noise-based sound than the tone-based sound. If an input of a predominantly noise-based sound is received, the method continues on to 612 to display a sound template for the selected type of noise and to adjust sound template parameters in accordance. For example, the method includes presenting the user with a white noise sound, a pink noise sound, and/or a broad band noise sound. In one example, a patient may be presented with two different noise based sounds and then be able to use a slide bar to select whether the tinnitus sound sounds more like a first sound or a second sound. It should be appreciated that the sound may be selected for the left ear or the right ear or both.

If the noise-based sound is not predominantly selected, the method continues on to 614 to determine if all noise types have been played. If not, then at 616, the method continues to present the user with another type of tinnitus therapy sound, such as a pure tone sound and/or a cricket sound. The method then returns to 610. Other methods of presenting the different sound types (e.g., templates) to a user are possible and may include presenting the sound templates in different combinations and/or orders.

Following the presentation of the tinnitus therapy sound template, the user interface of the healthcare professional's device will display a prompt to the user confirming the tinnitus therapy sound template selection. For example, confirming the tinnitus therapy sound template selection may include selecting whether the selected sound template is similar to the patient's perceived tinnitus. For example, after playing a white noise (at 608), it may be determined if a white noise sound was selected by the user, such as would occur if the presented white noise sound resembles a patient's perceived tinnitus. If a white noise sound is selected as a tinnitus sound similar to that of the patient's, a white noise sound template is displayed. In one example, upon selection of a tinnitus therapy sound template, a tinnitus sound, corresponding to the selection, will be presented to the user, such as in the form of a graphical user interface with a donut chart displaying the specific composition of the corresponding template. Following the presentation of the tinnitus therapy sound template, a user interface will display a prompt to the user confirming the tinnitus therapy sound template selection (e.g. white noise sound template). Once the tinnitus therapy sound template is selected, the user interface will display the tinnitus therapy sound template on the tinnitus therapy sound screen.

The method likewise continues to play each different noise type and receiver user input until all the noise types available have been provided to the user. At 618, based on the user input following the presenting of a specific noise type, the therapy sound is updated based on the selected templates. It may also be determined if an amplitude modulated sine wave template is selected. If the amplitude modulated sound template is selected, a user interface will display the amplitude modulated sine wave template. A user may then adjust an intensity and/or additional sound parameters of the sine modulated sine wave template. After any user inputs or adjustments, the method may include finalize the tinnitus therapy sound including the amplitude modulated sine wave template.

Following the selection, the details of the therapy sound are displayed to the patient and/or the healthcare professional as a graphical user interface. The graphical user interface may be a dashboard, such as shown at FIGS. 2-3. The GUI may display the current saved tinnitus therapy with intensities of each sound selected above the hearing threshold. The simplified interface may display a color gradient with a dark color corresponding to a high sound intensity and a light color corresponding a low sound intensity. Alternatively, the interface may display a donut chart with area-proportioned sectors corresponding to the different noises and their composition in the therapy sound.

An individual patient's perceived tinnitus may incorporate a plurality of tinnitus sounds; therefore, the method of FIG. 6 may be repeated until all templates have been selected. For example, a patient's perceived tinnitus may have sound characteristics of a combination of tinnitus sounds including white noise and broad band noise, white noise and pure tone, or pure tone and broad band noise. In yet another example, the patient's perceived tinnitus may include sound characteristics of two or more tinnitus sounds including two or more of white noise, pink noise, broad band noise, pure tone, amplitude modulated sine wave, and cricket. Additionally, the tinnitus therapy sound generated based on the selected tinnitus therapy sound templates may contain different proportions of the selected sound templates. For example, a generated tinnitus therapy sound may contain both pure tone and cricket sound components, but the pure tone component may make up a larger amount (e.g., 70%) of the combined tinnitus therapy sound. As such, two or more tinnitus therapy sound templates may be selected during the template selection process. In one example, a first tinnitus therapy sound template may include a white noise sound and a second tinnitus therapy sound template selection may include a pure tone sound. In another example, a first tinnitus therapy sound template may include a broad band noise sound template and a second tinnitus therapy sound template may include a white noise sound template. In another example, the first tinnitus therapy sound template may include a pure tone sound and a second tinnitus therapy sound template may include a broad band noise sound. In another example, a first tinnitus therapy sound template may include a cricket noise sound and a second tinnitus therapy sound template may include a white noise sound template.

In an additional example, a first tinnitus therapy sound template may include a pure tone sound template, a second tinnitus therapy sound template may include a broad band noise sound template, and a third tinnitus therapy sound template may include a white noise sound template. In another example, a first tinnitus therapy sound template may include a cricket noise sound template, a second tinnitus therapy sound template may include a broad band noise template, and a third tinnitus therapy sound template may include a white noise sound template. In an additional example, a first tinnitus therapy sound template may include a white noise sound template, a second tinnitus sound template may include a pure tone sound template, a third tinnitus therapy sound template may include a broad band noise template, and a fourth tinnitus therapy sound template may include a cricket noise sound template. After receiving one or more tinnitus therapy template selections, the selected tinnitus therapy template(s) may then be individually or simultaneously adjusted, to create the tinnitus therapy sound.

Turning now to FIG. 7, a method 700 for updating a GUI representing tinnitus sound therapy data in real-time is shown. The method enables more efficient tracking of a patient's progress by both the patient and their HCP.

At 702, the method includes isolating sound data from the selected tinnitus therapy sound. This may be the tinnitus therapy sound presented to a patient during a current therapy session. As discussed earlier, the therapy sound includes a specific combination of noises, including a modulation of their volumes, intensities, and frequencies, to generate a sound that best matches the patient's specific tinnitus sound. As such, it is expected that over time (e.g., over consecutive sessions), the noise volume for a specific sound will reduce. For example, if a patient's tinnitus largely matches cricket noise, then over prolonged exposure to cricket noise, there may be desensitization and the perceived cricket noise component of a patient's tinnitus sound is expected to reduce. This can be easily visually monitored via the changes and updates to chart displayed on the GUI.

Isolating the sound data includes, at 704, determining the identity of each type of noise in the therapy sound. For example, the composition of cricket noise, white noise, pink noise, etc., in the therapy sound for the patient may be retrieved. At 706, a percentage and sound intensity for each type of noise may be determined.

At 708, the method includes generating a graphical representation of the sound data from the tinnitus therapy sound. For example, the sound may be depicted as a donut chart including sectors representing each noise in the therapy sound. At 710, each type of noise may be depicted as a different sector on the pie chart or donut chart. At 712, an area of each sector may be adjusted to represent the noise contribution and/or noise intensity. For example, if a therapy sound includes equal parts of 4 different noises, then the chart may include 4 sectors of equal area. Further, at 714, the shading of each sector may be adjusted based on the noise intensity (e.g., volume). The intensity of each sound in the noise may be reflected as a color (or grayscale) gradient for each sector. The color gradients may be any color identified in the visible electromagnetic radiation spectrum. The color gradient may have different patterns. The color gradient may also be a black and white gradient. For example, as the cricket noise volume in the perceived tinnitus sound experienced by the patient decreases, the intensity or darkness of the sector representing the cricket noise may be reduced. Thus, an improvement in the patient's perceived tinnitus may be easily inferred by a HCP responsive to one or more of a decrease in the area of the cricket sector and a lightening of the cricket sector.

At 715, the method includes receiving user input regarding each noise type during sound matching of tinnitus therapy. That is, upon presenting the tinnitus therapy sound at a given therapy session, user input may be sought from the patient. The user may indicate, for example, if an intensity or volume of a provided sound matches their tinnitus sound. With reference to the sound survey of FIGS. 5-6, each time a noise is played for the user, the user is able to indicate whether the noise matches the noise in their perceived tinnitus. Over the course of the therapy, properties of each noise in the therapy sound are expected to change. For example, a specific noise may become louder or softer. Still other examples include changes in a specific noise's amplitude or frequency.

At 716, the method includes updating the graphical representation of the sound data based on the user input. This includes, at 718, reducing the area and/or intensity of a sector corresponding to a given noise responsive to a reduction in the volume of that noise during the therapy session. At 720, the area and/or intensity of a sector corresponding to a given noise may be increased responsive to an increase in the volume of that noise during the therapy session.

Each sound intensity maybe be assigned a pre-determined color such that the darker the color gradient, the higher the sound intensity. Further, when a sound intensity falls below a minimum threshold level, the darkness of the color may not be detectable. In one example, the minimum threshold level may be the hearing threshold determined by the audiogram data or hearing test. The darkness of the color may correlate with the sound intensity linearly. Thus, when the sound intensity falls below the hearing threshold, that sector may be removed from the GUI.

FIG. 8 shows an example GUI update. An initial GUI 800 may be generated during therapy session #12. Therein, the sound contribution of cricket noise, wave noise, white noise, and pure tone may be the same, as indicated by 4 sectors of the same area. This noise is presented to the user during the therapy session. However, during the session, the user may indicate that the volume of the wave noise in their perceived tinnitus has reduced. Upon matching, the wave noise is determined to have dropped below the user's hearing threshold. This results in a new therapy sound being generated, which is to be used for session #13. The updated GUI 810 removes the wave noise sector since the sound intensity fell below the hearing threshold, and the remaining sector areas are adjusted in accordance. In alternate examples, instead of adjusting the area, a shading of the wave sector may have been reduced, to make the sector lighter, in proportion to the reduction in volume perceived by the user.

In this way, a tinnitus therapy may be tracked and therapy data may be presented via a GUI. The visualization enables a user to more easily update and adjust a patient's tinnitus sound match and/or therapy parameters of the tinnitus sound match. More specifically, as one example, adjusting the tinnitus therapy may include changing one or more sound parameters of the tinnitus sound match. For example, intensity, frequency, or other sound parameters of one or more sound templates included in the tinnitus sound match may be adjusted. In another example, a new template may be added to the tinnitus sound match or another sound template may be removed from the tinnitus sound match. In another example, a new tinnitus sound match may be created including a different sound template than the original sound match. In this way, a user may utilize tracked data to guide tinnitus therapy changes in order to better treat the patient. By tracking patient therapy data over time via a GUI, and presenting the tracked data to a user via the GUI, changes to (or the evolution of) a patient's tinnitus may be identified. Further, by adjusting the patient's tinnitus therapy (including the tinnitus sound match) based on the tracked therapy data, a more effective tinnitus treatment may be prescribed to the patient. As a patient's tinnitus continues to evolve over time, the tinnitus therapy may be updated to match a patient's perceived tinnitus sound and further reduce the patient's tinnitus.

In alternate embodiments, the methods presented for generating a tinnitus therapy sound or match may also be used to generate a sound or match for therapy of other neurological disorders. For example, the generated audio sound may be at least partially used for treating neurological disorders such as dizziness, hyperacusis, misophonia, Meniere's disease, auditory neuropathy, autism, chronic pain, epilepsy, Parkinson's disease, and recovery from stroke. In this embodiment, sound templates may be adjusted based on patient data, the patient data being specific to the neurological disorder. In some examples, different combinations of the above described sound templates may be used to generate an audio sound or match for one of the neurological disorders.

An example of a method comprises displaying a graphical user interface including a visual representation of real-time changes to a therapy sound presented to a user during a tinnitus therapy, the real-time changes based on user input.

A first example of the method further includes where the graphical user interface includes a donut chart with a plurality of sectors, each of the plurality of sectors representative of a distinct noise included in the therapy sound.

A second example of the method, optionally includes each of the previous examples, further includes where the displaying includes reducing an area of a given sector responsive to the user input indicating a reduction in an intensity of a corresponding noise.

A third example of the method, optionally includes each of the previous examples, further includes where each of the plurality of sectors has a distinct color, and wherein the displaying includes reducing a brightness of the color of a given sector responsive to the user input indicating a reduction in an intensity of a corresponding noise.

A fourth example of the method, optionally including each of the previous examples, further includes where the displaying includes displaying on a user device, the user device including a patient device and a healthcare provider device communicatively coupled to each other.

An embodiment of a method, comprises displaying a graphical user interface including a visual representation of real-time changes to a therapy sound presented to a user related to a tinnitus therapy, the real-time changes based on changes in a matched sound profile of a user's tinnitus.

A first example of the method, further includes where the visual changes include reducing a brightness of a first component of the matched sound profile responsive to the user indicating a lower volume of the first component in subsequent sound matches by the user.

A second example of the method, optionally including each of the previous examples, further includes where the visual changes include reducing a brightness of a second component of the matched sound profile responsive to the user indicating a lower volume of the second component in subsequent sound matches by the user relative to a third component of the sound match.

A third example of the method, optionally including each of the previous examples, further includes where the visual changes include reducing a size of a first component of the matched sound profile relative to a second component of the matched sound responsive to the user indicating a lower volume of the first component in subsequent sound matches by the user relative to the second component.

An embodiment of a system comprises a tinnitus therapy treatment system comprising a patient device and a healthcare professional device, wherein each of the patient device and the healthcare professional device comprise a display configured to display a graphical user interface (GUI), and a controller with computer-readable instruction stored on memory thereof that when executed enable the controller to receive user inputs from a patient or a healthcare professional, wherein the inputs from the patient or the healthcare professional are in response to one or more prompts displayed on the display and sounds played via an audio device and adjust a future tinnitus therapy session in response to the inputs.

A first example of the system, further includes where the patient device and the healthcare professional device are communicatively coupled such that data is transferred between the patient device and the healthcare professional device, and where the data includes a tinnitus therapy session and inputs from the patient and the healthcare professional.

A second example of the system, optionally including any of the previous examples, further includes where the display is a touchscreen, and where the audio device is one or more of an in-ear headphone, an over-ear headphone, a behind-neck headphone, a wired headphone, a wireless headphone, and a speaker.

A third example of the system, optionally including any of the previous examples, further includes where sounds played include one or more active sounds played in equal or unequal amounts.

A fourth example of the system, optionally including any of the previous examples, further includes where a tinnitus therapy session includes a plurality of active sounds, and where the user inputs adjust a volume adjustment, a frequency adjustment, a timbre adjustment, a Q factor adjustment, a vibrato adjustment, a reverberation adjustment, and/or a white noise edge enhancement adjustment.

A fifth example of the system, optionally including any of the previous examples, further includes where an adjustment to a first active sound the plurality of active sounds is equally distributed to n remaining active sounds of the plurality of active sounds.

A sixth example of the system, optionally including any of the previous examples, further includes where the user inputs select one or more active sounds for a first therapy session, and where the user inputs adjust the one or more active sounds for a second therapy session subsequent the first therapy session.

A seventh example of the system, optionally including any of the previous examples, further includes where the second therapy session comprises fewer active sounds than the first therapy session, and where a contribution of each active sound is illustrated as a sector of a donut, wherein each sector combines to shape the donut, and where the donut is displayed via the display to the user.

An eighth example of the system, optionally including any of the previous examples, further includes where each of the first and second therapy sessions are saved and configured to be used during future therapy sessions, further comprising where a plurality of templates comprising various combinations of active sounds are selectable for future therapy sessions.

A ninth example of the system, optionally including any of the previous examples, further includes where the one or more active sounds include a white noise sound, a pink noise sound, a pure tone sound, a broad band noise sound, a cricket noise sound, an amplitude modulated sine wave, and/or a combined tone sound.

A tenth example of the system, optionally including any of the previous examples, further includes where the future tinnitus therapy session comprises adjusted contributions and volumes for a plurality of active sounds used in the future tinnitus therapy session.

The healthcare professional's device may allow a healthcare provider to manage one or more patients or users. For example, the healthcare professional's device may include one or more administrative or patient management screens (e.g., user interfaces or displays) that enabled the healthcare provider to select and then manage data of one or more patients. For example, a patient may be selected and statistics (e.g., tracked data) may be provided to show a patient's progress or data tracking for a single session or a plurality of sessions. Information regarding the patient or patient's tinnitus therapy may be inputted, tracked and in some examples linked with other records or databases, including but not limited to digital medical records.

1. A method, comprising:
displaying a graphical user interface including a visual representation of real-time changes to a therapy sound presented to a user during a tinnitus therapy, the real-time changes based on user input.

2. The method of claim 1, wherein the graphical user interface includes a donut chart with a plurality of sectors, each of the plurality of sectors representative of a distinct noise included in the therapy sound.

3. The method of claim 1, wherein the displaying includes reducing an area of a given sector responsive to the user input indicating a reduction in an intensity of a corresponding noise.

4. The method of claim 1, wherein each of the plurality of sectors has a distinct color, and wherein the displaying includes reducing a brightness of the color of a given sector responsive to the user input indicating a reduction in an intensity of a corresponding noise.

5. The method of claim 1, wherein the displaying includes displaying on a user device, the user device including a patient device and a healthcare provider device communicatively coupled to each other.

6. A method, comprising:
displaying a graphical user interface including a visual representation of real-time changes to a therapy sound presented to a user related to a tinnitus therapy, the real-time changes based on changes in a matched sound profile of a user's tinnitus.

7. The method of claim 6, wherein the visual changes include reducing a brightness of a first component of the matched sound profile responsive to the user indicating a lower volume of the first component in subsequent sound matches by the user.

8. The method of claim 7, wherein the visual changes include reducing a brightness of a second component of the matched sound profile responsive to the user indicating a lower volume of the second component in subsequent sound matches by the user relative to a third component of the sound match.

9. The method of claim 6, wherein the visual changes include reducing a size of a first component of the matched sound profile relative to a second component of the matched sound responsive to the user indicating a lower volume of the first component in subsequent sound matches by the user relative to the second component.

10. A system comprising:
a tinnitus therapy treatment system comprising a patient device and a healthcare professional device, wherein each of the patient device and the healthcare professional device comprise a display configured to display a graphical user interface (GUI); and
a controller with computer-readable instruction stored on memory thereof that when executed enable the controller to:
receive user inputs from a patient or a healthcare professional, wherein the inputs from the patient or the healthcare professional are in response to one or more prompts displayed on the display and sounds played via an audio device; and
adjust a future tinnitus therapy session in response to the inputs.

11. The system of claim 10, wherein the patient device and the healthcare professional device are communicatively coupled such that data is transferred between the patient device and the healthcare professional device, and where the data includes a tinnitus therapy session and inputs from the patient and the healthcare professional, wherein the patient device and the healthcare professional device are a Wi-Fi enabled device including one or more of a cell phone, personal computer, tablet, smart phone, and a smart listening device.

12. The system of claim 10, wherein the display is a touchscreen, and where the audio device is one or more of an in-ear headphone, an over-ear headphone, a behind-neck headphone, a wired headphone, a wireless headphone, a hearing aid, and a speaker.

13. The system of claim 10, wherein sounds played include one or more active sounds played in equal or unequal amounts.

14. The system of claim 11, wherein a tinnitus therapy session includes a plurality of active sounds, and where the user inputs adjust a volume adjustment, a frequency adjustment, a timbre adjustment, a Q factor adjustment, a vibrato adjustment, a reverberation adjustment, and/or a white noise edge enhancement adjustment.

15. The system of claim 14, wherein an adjustment to a first active sound the plurality of active sounds is equally distributed to n remaining active sounds of the plurality of active sounds.

16. The system of claim 10, wherein the user inputs select one or more active sounds for a first therapy session, and where the user inputs adjust the one or more active sounds for a second therapy session subsequent the first therapy session.

17. The system of claim 16, wherein the second therapy session comprises fewer active sounds than the first therapy session, and where a contribution of each active sound is illustrated as a sector of a donut, wherein each sector combines to shape the donut, and where the donut is displayed via the display to the user.

18. The system of claim 17, wherein each of the first and second therapy sessions are saved and configured to be used during future therapy sessions, further comprising where a plurality of templates comprising various combinations of active sounds are selectable for future therapy sessions.

19. The system of claim 16, wherein the one or more active sounds include a white noise sound, a pink noise sound, a pure tone sound, a broad band noise sound, a cricket noise sound, an amplitude modulated sine wave, and/or a combined tone sound.

20. The system of claim 10, wherein the future tinnitus therapy session comprises adjusted contributions and volumes for a plurality of active sounds used in the future tinnitus therapy session.
